(19) United States
(12) Patent Application Publication
(10) Pub. No.: US 2018/0008817 A1
(43) Pub. Date: Jan. 11, 2018

(54) BIOLOGICAL ELECTRODE AND BIOLOGICAL ELECTRODE-EQUIPPED WEARING TOOL

(71) Applicant: SMK Corporation, Tokyo (JP)

(72) Inventors: Koichiro EJIRI, Kanagawa (JP); Haruhiko KONDO, Kanagawa (JP)

(21) Appl. No.: 15/623,404

(22) Filed: Jun. 15, 2017

(30) Foreign Application Priority Data
Jul. 11, 2016 (JP) 2016-136552

(51) Int. Cl. A61N 1/04; A41D 19/00; A41B 11/00; A61B 5/00; A61B 5/04

(52) U.S. Cl. CPC A61N 1/0484; A61B 5/6807; A61B 5/6806; A61B 5/04; A41B 11/00; A41D 19/0027; A41B 2400/32; A41D 2400/32

(57) ABSTRACT

Provided is a biological electrode and a biological electrode-equipped wearing tool, in which a contact surface having a certain surface area is suitably brought into intimate contact with a living body, and a suitable electrical distribution is obtained. The biological electrode includes: a thin-plate-like or sheet-like wiring substrate having a lead wire portion to be connected with equipment; a plurality of electrode convex portions disposed on a surface of the wiring substrate in a state of being electrically connected with the lead wire portion; and a conductive cloth portion which is superimposed on the wiring substrate through the electrode convex portions. The conductive cloth portion is to be contacted with the surface of a living body, and electrically connected to the living body.

### CROSS REFERENCE TO RELATED APPLICATION

The contents of the following Japanese patent application are incorporated herein by reference,

Japanese Patent Application No. 2016-136552 filed on Jul. 11, 2016.

### FIELD

The present invention relates to a biological electrode and a biological electrode-equipped wearing tool, which are to be contacted with a living body such as the human body mainly for stimulating muscles or measuring physical information.

### BACKGROUND

There have been known a therapeutic apparatus in which an electrode is brought into contact with a desired position of the human body to provide electrical stimuli to muscles through the electrode, such as a low-frequency therapeutic device.

A biological electrode used for such a therapeutic apparatus has a problem in that a commonly-used metal electrode is unlikely to ensure sufficient contact surface areas with the human body when brought into contact with the skin, and the contact surface cannot follow the change of the skin shape associated with the movement of the human body.

The contact surface areas with the human body and the properties of following the change of the skin shape have been ensured by placing gauze impregnated with a conductive medium such as a salt solution between the metal electrode body and the skin, and fixing the gauze and the electrode body with a band or the like.

Also, a biological electrode for wearable apparatuses, in which a planar electrode having a certain surface area is formed by weaving a conductive cloth to clothes and is connected to a connection terminal, and the electrode is brought into a desired position of a human body by wearing the cloths so that it follows the skin shape has been developed (see JP-A-2005-349021, for example).

### SUMMARY

### Technical Problem

However, the above-described technologies had a risk that electric currents supplied from the electrode body through the conductive medium such as a salt solution may be dispersed, inhibiting stimuli from being efficiently provided to muscles.

Also, since the planar electrode constituted by a conductive cloth has a certain surface area, there was a risk that a long distance from the actually stimulated position to the connection terminal may cause electric resistance to increase, thereby reducing the output of electrical signals in that portion.

In view of such problems, an object of an embodiment according to the present invention is to provide a biological electrode and a biological electrode-equipped wearing tool, in which a contact surface having a certain surface area is suitably brought into intimate contact with a living body, and a suitable electrical distribution is obtained.

### Solution to Problem

For solving the above-described problems, a first aspect of the present invention is a biological electrode which is to be contacted with the surface of a living body and electrically connected to the living body. The biological electrode includes: a thin-plate-like or sheet-like wiring substrate having a lead wire portion to be connected with equipment; a plurality of electrode convex portions disposed on the surface of the wiring substrate in the state of being electrically connected with the lead wire portion; and a conductive cloth portion which is superimposed on the wiring substrate through the electrode convex portions. The conductive cloth portion is to be contacted with the surface of the living body.

A second aspect of the present invention is the biological electrode according to the first aspect, in which the lead wire portion includes connection terminals to be connected with equipment and terminal connection lead wires for connecting the connection terminals and the electrode convex portions.

A third aspect of the present invention is the biological electrode according to the first or second aspect, in which the lead wire portion includes inter-electrode connection lead wires for connecting the electrode convex portions to each other.

A fourth aspect of the present invention is the biological electrode according to any one of the first to third aspects, in which the wiring substrate is a flexible printed circuit board.

A fifth aspect of the present invention is a biological electrode-equipped wearing tool, which includes a biological electrode integrated with an insulating cloth portion that constitutes a wearing tool. The biological electrode is brought into intimate contact with the surface of a living body by wearing the wearing tool. The biological electrode includes: a thin-plate-like or sheet-like wiring substrate having a lead wire portion to be connected with equipment; a plurality of electrode convex portions disposed on the surface of the wiring substrate in the state of being electrically connected with the lead wire portion; and a conductive cloth portion which is superimposed on the wiring substrate through the electrode convex portions. The insulating cloth portion, the wiring substrate, the electrode convex portions, and the conductive cloth portion are integrated in a layered arrangement.

A sixth aspect of the present invention is the biological electrode-equipped wearing tool according to the fifth aspect, in which the wearing tool is clothes.

A seventh aspect of the present invention is the biological electrode-equipped wearing tool according to the fifth aspect, in which the wearing tool is an attachment tool having a shape of a band, a tube, socks, or gloves for covering muscles, joints or ligaments.

As described above, the biological electrode according to an embodiment of the present invention is a biological electrode to be contacted with the surface of a living body and electrically connected to the living body. The biological electrode includes: a thin-plate-like or sheet-like wiring substrate having a lead wire portion to be connected with equipment; a plurality of electrode convex portions disposed on the surface of the wiring substrate in the state of being electrically connected with the lead wire portion; and a conductive cloth portion which is superimposed on the wiring substrate through the electrode convex portions. The conductive cloth portion is to be brought into intimate contact with the surface of the living body. Accordingly, the biological electrode can suitably follow the variation of the surface of the living body in an intimate contact state while ensuring a certain contact surface area with the living body. Also, multipolarity enables favorable electrical distribution to be obtained. Thus, dispersion of electrical currents and partial decrease of outputs can be suitably prevented.

Also, in an embodiment of the present invention, the lead wire portion includes connection terminals to be connected with equipment and terminal connection lead wires for connecting the connection terminals and the electrode convex portions, or inter-electrode connection lead wires for connecting the electrode convex portions to each other. Therefore, a circuit depending on an application can be configured in the electrode portion contacted with a living body.

Furthermore, in an embodiment of the present invention, the wiring substrate may be a flexible printed circuit board. Accordingly, the biological electrode can suitably and flexibly follow the deformation of the conductive cloth portion associated with the movement of a living body.

In an embodiment of the present invention, the biological electrode-equipped wearing tool includes a biological electrode integrated with an insulating cloth portion which constitutes a wearing tool, and the biological electrode is brought into intimate contact with the surface of a living body by wearing the wearing tool. The biological electrode includes: a thin-plate-like or sheet-like wiring substrate having a lead wire portion to be connected with equipment; a plurality of electrode convex portions disposed on the surface of the wiring substrate in the state of being electrically connected with the lead wire portion; and a conductive cloth portion which is superimposed on the wiring substrate through the electrode convex portions. The insulating cloth portion, the wiring substrate, the electrode convex portions, and the conductive cloth portion are integrated in a layered arrangement. Therefore, the biological electrode can be brought into intimate contact with a desired position of a living body. Furthermore, the biological electrode can suitably follow the variation of the surface of a living body in an intimate contact state while ensuring a certain contact surface area with the living body. Also, multipolarity enables a favorable electrical distribution to be obtained. Thus, dispersion of electrical currents and partial decrease of outputs can be suitably prevented.

Also, since the wearing tool is clothes in an embodiment of the present invention, the biological electrode can be easily brought into contact with the surface of a living body by wearing the clothes. Furthermore, since the wearing tool is alternatively an attachment tool having a shape of a band, a tube, socks, or gloves for covering muscles, joints or ligaments, the biological electrode can be brought into intimate contact with a local position of a living body according to the purpose.

### BRIEF DESCRIPTION OF DRAWINGS

FIG. 1 is a front view illustrating an example of a biological electrode-equipped wearing tool according to an embodiment of the present invention;

FIG. 2 is a partially enlarged cross-sectional view illustrating a biological electrode in FIG. 1;

FIG. 3A is a planar view illustrating an example of a wiring substrate;

FIG. 3B is a schematic circuit diagram when the wiring substrate is used;

FIG. 4A is a planar view illustrating another example of a wiring substrate;

FIG. 4B is a schematic circuit diagram when the wiring substrate is used;

FIG. 5A is a planar view illustrating further another example of a wiring substrate; and

FIG. 5B is a schematic circuit diagram when the wiring substrate is used.

### DESCRIPTION OF EMBODIMENTS

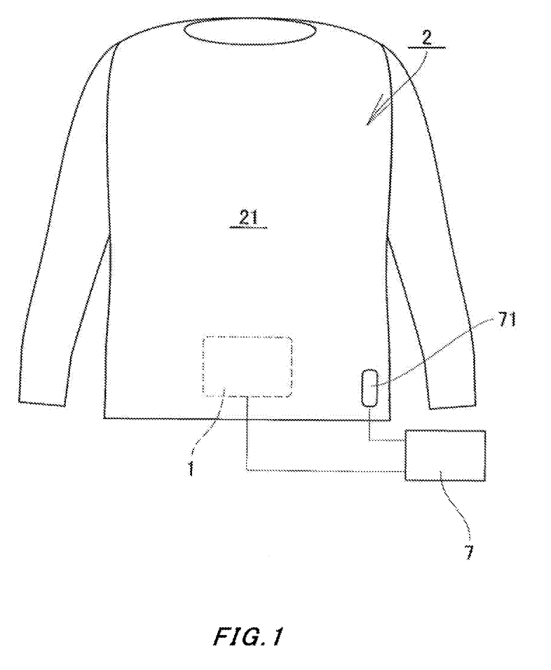

Next, embodiments of a biological electrode and a biological electrode-equipped wearing tool according to the present disclosure will be described based on examples illustrated in FIG. 1 to FIG. 5B. It is noted that, in the drawings, reference numeral 1 indicates a biological electrode, and reference numeral 2 indicates a biological electrode-equipped wearing tool.

As illustrated in FIG. 1, the biological electrode-equipped wearing tool 2 constitutes an apparatus which includes the biological electrode 1 integrated with an insulating cloth portion 22 that constitutes clothes 21 and which has an electric circuit through a living body by electrically connecting the biological electrode 1 with equipment 7. It is noted that, in FIG. 1, reference numeral 71 indicates a GND electrode to be contacted with a living body 6 or other portions.

The insulating cloth portion 22 has the shape of a stretchable and flexible cloth formed with chemical fibers such as polyester. The clothes 21 have an embodiment which suits an application such as a shirt, a band, and socks formed with the insulating cloth portion 22, and are to be brought into intimate contact with the living body 6 by wearing the clothes 21.

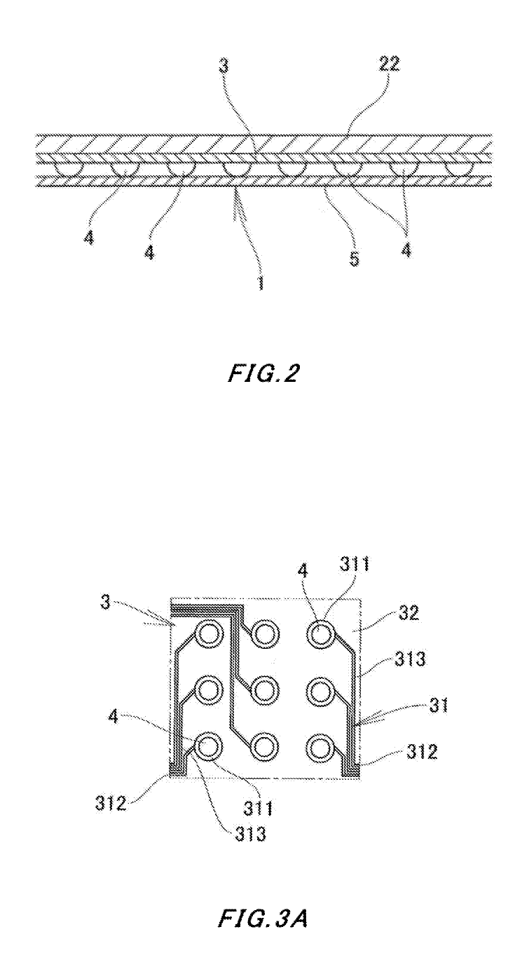

The biological electrode 1 includes, as illustrated in FIG. 2, a thin-plate-like or sheet-like wiring substrate 3 having a lead wire portion 31, a plurality of electrode convex portions 4 disposed on the surface of the wiring substrate 3, and a conductive cloth portion 5 which is superimposed on the wiring substrate 3 through the electrode convex portions 4. The conductive cloth portion 5 is to be brought into intimate contact with the surface of the living body 6.

The wiring substrate 3 is constituted by a flexible printed circuit board (FPC), and includes the lead wire portion 31 formed on one surface of an insulating sheet body 32. The lead wire portion 31 includes copper foil and the like, and constitutes an electrical circuit. This lead wire portion 31 is to be electrically connected to the equipment 7.

The sheet body 32 includes insulating resin such as polyimide, has a shape of a thin sheet, and exhibits pliability and flexibility.

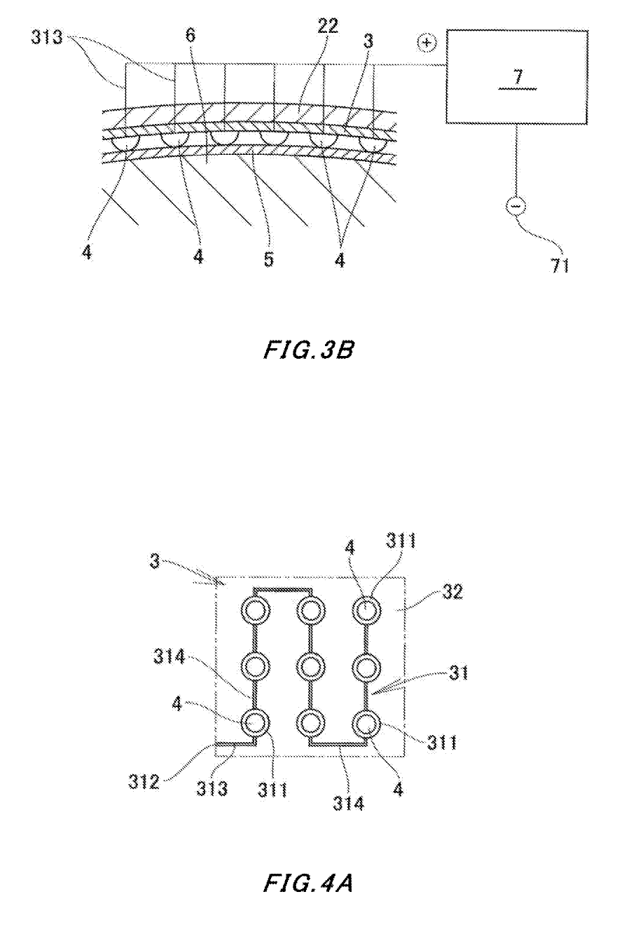

The lead wire portion 31 includes, for example, as illustrated in FIGS. 3A and 3B, implementation pattern portions 311 to which the electrode convex portions 4 are implemented, connection terminal portions 312 to be connected with the electronic equipment 7, and terminal connection lead wires 313 for connecting the implementation pattern portions 311 and the connection terminal portions 312. The electrode convex portions 4 are to be connected in parallel to the equipment 7.

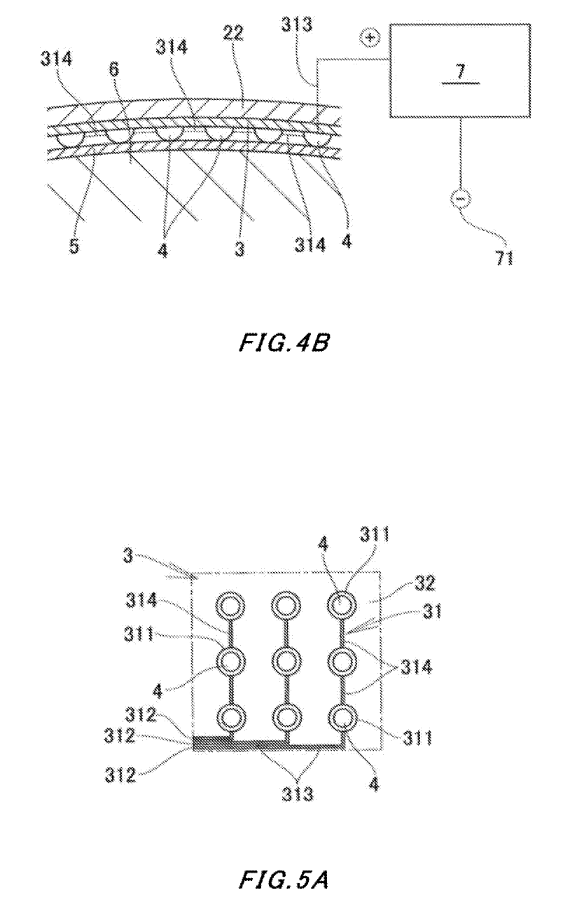

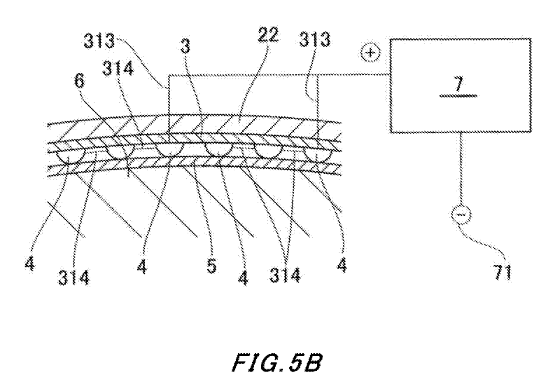

It is noted that an embodiment of the lead wire portion 31 is not limited to the embodiment indicated in FIGS. 3A and 3B. For example, as illustrated in FIGS. 4A and 4B, inter-electrode connection lead wires 314 for connecting the implementation pattern portions 311, that is, the electrode convex portions 4, may be provided such that all of the electrode convex portions 4 are connected in series. Alternatively, as illustrated in FIGS. 5A and 5B, electrode convex portion rows each including the plurality of electrode convex portions 4 connected in series via the inter-electrode connection lead wires 314 may be formed, and the electrode convex portion rows are connected in parallel to the connection terminal portions 312 through the terminal connection lead wires 313.

The electrode convex portions 4 include surfaces having been plated with conductive metal such as gold, and have a hemispherical shape. The electrode convex portions 4 are implemented to the implementation pattern portions 311 of the lead wire portion 31 in such a manner as to be spaced apart from each other on the surface of the wiring substrate 3, while being electrically connected to the lead wire portion 31.

The conductive cloth portion 5 is formed into the shape of a stretchable and flexible cloth or sheet with ultrathin chemical fibers such as nanofibers. Furthermore, conductive macromolecules are allowed to enter between the chemical fibers, so that the entire cloth portion has conductivity.

Also, the conductive cloth portion 5 includes a fixing unit that fixes the margin of the conductive cloth portion 5 to the insulating cloth portion 22. Since the conductive cloth portion 5 is fixed to the insulating cloth portion 22, the wiring substrate 3 and the electrode convex portions 4 are sandwiched between the insulating cloth portion 22 and the conductive cloth portion 5. Thus, the insulating cloth portion 22, the wiring substrate 3, the electrode convex portions 4 and the conductive cloth portion 5 are integrated in a layered arrangement.

The unit for fixing the conductive cloth portion 5 to the insulating cloth portion 22 is not particularly limited. For example, fibers which constitute the conductive cloth portion 5 and fibers which constitute the insulating cloth portion 22 may be knitted, or the margin of the conductive cloth portion 5 may be heat-sealed, sewn, or pasted.

This conductive cloth portion 5 is superimposed on the wiring substrate 3 in the state of being in contact with the electrode convex portions 4. Thus, the conductive cloth portion 5 and the wiring substrate 3 can follow each other to be deformed while being electrically connected through the electrode convex portions 4.

According to the biological electrode-equipped wearing tool 2 configured as described above, the insulating cloth portion 22 has stretchability. Therefore, the clothes 21 are brought into intimate contact with the surface of the living body 6 when worn. Accordingly, the biological electrode 1 integrated with the insulating cloth portion 22 is also brought into intimate contact with the living body 6.

At that time, the conductive cloth portion 5 made of fibers in the biological electrode 1 is in intimate contact with the surface of a living body. Thus, a favorable feel and a certain contact surface area with the living body 6 can be obtained.

Furthermore, in this biological electrode 1, the conductive cloth portion 5 has stretchability and flexibility. Therefore, the conductive cloth portion 5 follows the movement of the living body 6 and is deformed such that the state of being in intimate contact with the living body 6 is maintained. At the same time, since the wiring substrate 3 has pliability and flexibility, it can follow the deformation of the conductive cloth portion 5.

At that time, the wiring substrate 3 and the conductive cloth portion 5 are always in contact with each other through the electrode convex portions 4 in a multipolar manner. Therefore, a suitable electrical distribution can be obtained in any position of the conductive cloth portion 5.

It is noted that although an example in which a flexible printed circuit board (FPC) is used as the wiring substrate 3 has been described in the above-described example, any pliable and flexible thin-plate-like or sheet-like substrate other than an FPC may be used.

Also, although an example in which clothes such as a shirt is used as the wearing tool 2 has been described in the above-described example, an embodiment of the clothes is not limited to the above-described example, and may have a shape of a vest, a belly band, or the like.

Furthermore, an embodiment of the wearing tool 2 is not limited to clothes, and may be an attachment tool (a so-called supporter) having a shape of a band, a tube, socks, or gloves for covering muscles, joints or ligaments.

Also, although the biological electrode-equipped wearing tool 2 has been described in the above-described example, the biological electrode 1 according to an embodiment of the present invention can also be applied to another object which is used in the state of being in contact with a living body, such as a chair and a bed, in addition to the wearing tool indicated in the above-described examples.

Also, a living body is not limited to the human body, and may be animals such as pets and livestock.

1. A biological electrode to be contacted with a surface of a living body and electrically connected to the living body, comprising:
a thin-plate-like or sheet-like wiring substrate having a lead wire portion to be connected with equipment;
a plurality of electrode convex portions disposed on a surface of the wiring substrate in a state of being electrically connected with the lead wire portion; and
a conductive cloth portion which is superimposed on the wiring substrate through the electrode convex portions, wherein
the conductive cloth portion is to be contacted with the surface of the living body.

2. The biological electrode according to claim 1, wherein the lead wire portion includes connection terminals to be connected with equipment and terminal connection lead wires for connecting the connection terminals and the electrode convex portions.

3. The biological electrode according to claim 1, wherein the lead wire portion includes inter-electrode connection lead wires for connecting the electrode convex portions to each other.

4. The biological electrode according to claim 1, wherein the wiring substrate is a flexible printed circuit board.

5. A biological electrode-equipped wearing tool comprising a biological electrode integrated with an insulating cloth portion which constitutes a wearing tool, the biological electrode being brought into intimate contact with a surface of a living body by wearing the wearing tool, wherein
the biological electrode includes:
a thin-plate-like or sheet-like wiring substrate having a lead wire portion to be connected with equipment;
a plurality of electrode convex portions disposed on a surface of the wiring substrate in a state of being electrically connected with the lead wire portion; and
a conductive cloth portion which is superimposed on the wiring substrate through the electrode convex portions, and
the insulating cloth portion, the wiring substrate, the electrode convex portions, and the conductive cloth portion are integrated in a layered arrangement.

6. The biological electrode-equipped wearing tool according to claim 5, wherein the wearing tool is clothes.

7. The biological electrode-equipped wearing tool according to claim 5, wherein the wearing tool is an attachment tool having a shape of a band, a tube, socks, or gloves for covering muscles, joints or ligaments.
